(19) United States
(12) Patent Application Publication
(10) Pub. No.: US 2017/0311972 A1
(43) Pub. Date: Nov. 2, 2017

(54) Medical Device for Debridement of Tissue

(71) Applicant: Elvis Castillo-Garcia, Wesley Chapel, FL (US)

(72) Inventor: Elvis Castillo-Garcia, Wesley Chapel, FL (US)

(21) Appl. No.: 15/653,834

(22) Filed: Jul. 19, 2017

Related U.S. Application Data
(63) Continuation-in-part of application No. 15/142,139, filed on Apr. 29, 2016.
(60) Provisional application No. 62/491,610, filed on Apr. 28, 2017.

(51) Int. Cl. A61B 17/3211; A61B 17/3207; A61B 17/22; A61B 17/00; A61B 17/32

(52) U.S. Cl. CPC A61B 17/3211; A61B 17/320708; A61B 17/22; A61B 2017/320008; A61B 2017/00424

(57) ABSTRACT

The device comprises a generally cylindrically shaped body having an elongated base with a distal end and a proximal end, and is conformed to be received by a fingertip of a medical practitioner. A plurality of prongs for debridement scrapping are formed on an outer surface of the base. A pair of arcuately shaped portions are integrally formed with the base to secure the device to any finger of a medical practitioner.

### FIELD OF THE INVENTION

The present invention relates to a medical device for wound debridement, namely the removal of dead, damaged or infected tissue by cutting or by scrapping to assist on the alteration of tissue from chronic phase to acute phase.

### BACKGROUND OF THE INVENTION

Debridement is the removal of dead, damaged or infected tissue in wounds to improve the healing of the remaining healthy tissue. Surgical instruments, such as curettes, scalpels, scissors and forceps, are normally used by medical practitioners to debride a wound. These instruments present a disadvantage in that access to the entire area of treatment is limited.

The current invention is directed to a debridement medical device mounted on a forefinger of a practitioner to access areas not available to conventional surgical tools, such as curettes due to their rigid, structure. Metal prongs on a base facilitate mild debridement of a wound in areas that cannot be accessed and facilitate crosshatching thicker eschar (a slough or piece of dead tissue) without having to use a blade.

The current invention is convenient to use due to its ability to access tunnels or tunneled wounds, and wounds with undermining. Time will be saved in healing chronic or acute wounds with dead, damaged or infected tissue.

### SUMMARY OF THE INVENTION

The general purpose of the present invention, which will be described subsequently in greater detail, is to provide a new and improved wound debridement medical device.

In an embodiment the device comprises a generally cylindrically shaped body distal end and a proximal end, and is conformed to be received by a fingertip of a medical practitioner. A plurality of metal prongs for debridement scrapping are positioned on an outer surface, and along a bottom surface of the body.

A pair of arcuately shaped holding portions having an inner surface and an outer surface are integrally formed with the base. The portions are spaced apart and secure the device to any finger of a medical practitioner.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a side elevational view of a first embodiment of the present invention mounted on the forefinger of a medical practitioner.

FIG. 2 is a front elevational view of the present invention.

FIG. 3 is a rear elevational view of the present invention.

FIG. 4 is a top plan view of the present invention.

FIG. 5 is a bottom plan view of the present invention.

FIG. 6 is a side elevational view of the present invention.

### DETAILED DESCRIPTION OF THE INVENTION

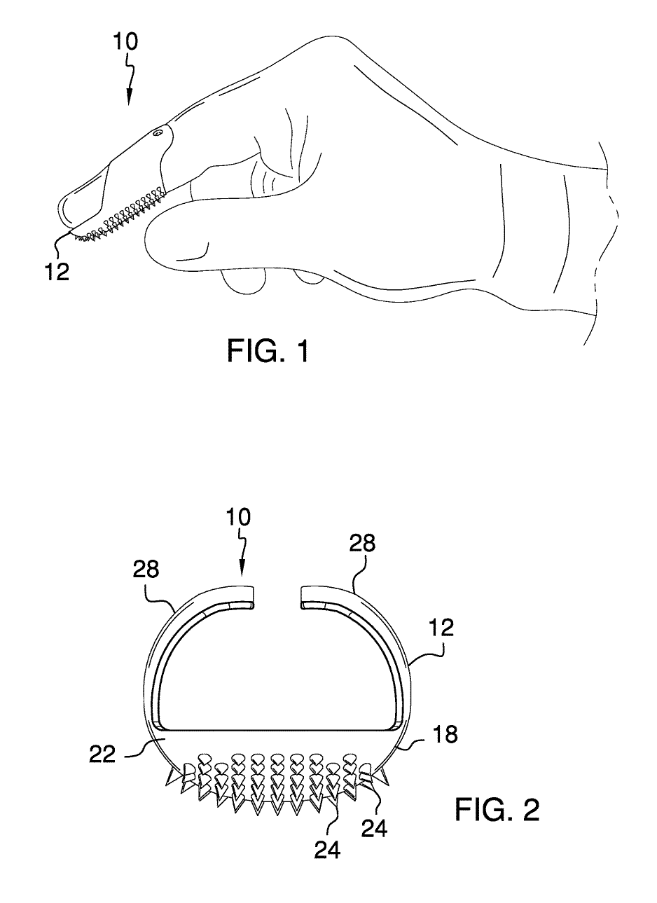

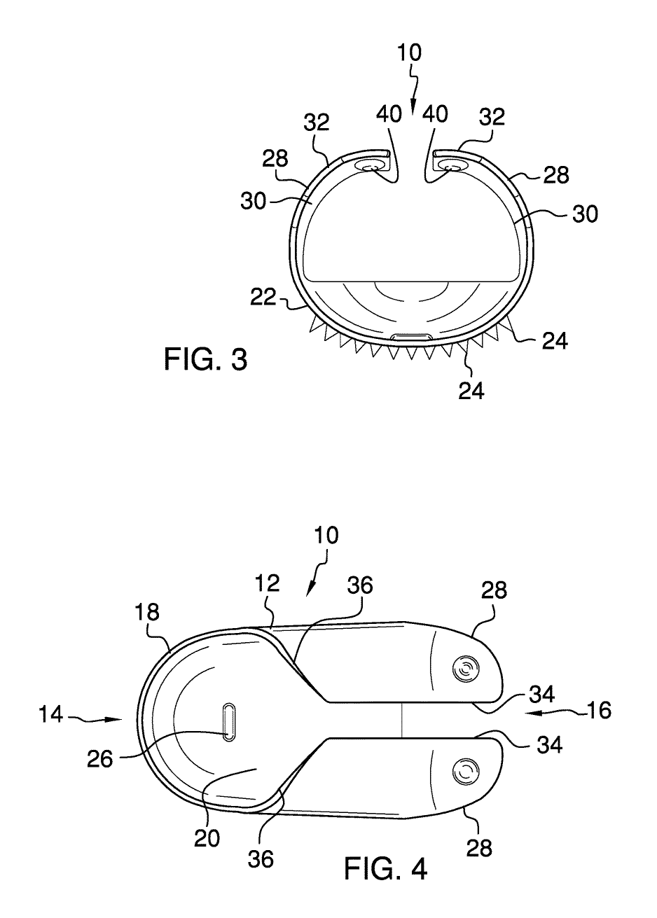

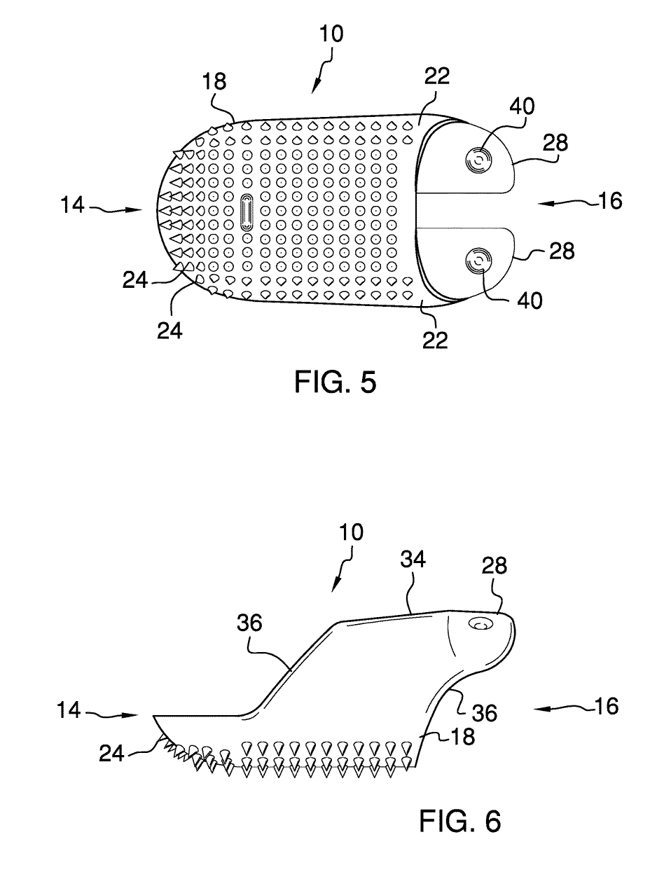

Referring generally to the FIGS. 1-6, an embodiment 10 of the present invention is illustrated. A generally cylindrically shaped body 12 has a first or distal end 14 and a second or proximal end 16, and is conformed to be received by a fingertip of a medical practitioner. The body 12 comprises an elongated base 18 having a generally concave inner surface 20 longitudinally extending from the distal end 14 of the body 12 to the proximal end 16 of the body 12, and having a generally curvilinear shaped outer surface 22. A plurality of prongs 24 with sharpened distal ends are integrally formed and positioned along the outer surface 22 of the base 18. The inner surface 20 is generally adapted in shape to the contour of a finger.

The prongs 24 extend vertically from the outer surfaces 22 and are generally conically shaped. The sharpened ends of the prongs 24 are directed towards a scrapping action in all directions, and also facilitate mild debridement in areas not easily accessible by a conventional curette, due to the rigid shape of the curette. The shape of the device 10 allows the practitioner not only to debride shallow areas, but also to debride tunnels and underminings. The prongs 34 are not limited to any specific, configurations, and come in a variety of oriented arrangements, indentations and contact surface prominences.

A ridge 26 is integrally formed medially on the inner surface 20 relatively towards the distal end 14 of the device 10. The ridge 26 grips snugly against the finger of the practitioner helping to prevent detachment during, use.

A first arcuately shaped portion 28 and, a second arcuately shaped portion 28, each having an inner surface 30 and an outer surface 32, are integrally formed with the base 18 at opposed sides of the base 18, preferably at the proximal end 14 of the device 10. The portions 28 each have a front edge 34 facing each other, and side edges 36. A first knurl 40 is formed on the inner surface 30 near the front edge 34 of the first portion 28, and a second knurl 40 is formed on the inner surface near the front edge 34 of the second portion 28. The knurls 40 abut the finger of a practitioner during used and helps retain the finger in engagement with the device. The portions 28 are flexible and can be spread apart against a restoring force, so as to adapt the device to different finger widths.

The device is composed of suitable resilient and flexible material, such as plastic, stainless steel, nickel plating or formed by additive manufacturing metal fabrication technology, such as direct metal laser sintering (DMLS).

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims attached.

I claim:

1. A medical device for debridement of tissue, comprising in combination:
a generally cylindrical shaped body having a base with a generally concave inner surface and an outer surface;
a plurality of prongs, each having a sharpened distal end, extending vertically from an outer surface of the base; and
a first arcuately shaped portion and a second arcuately shaped portion integrally formed with the base at opposed sides of the base for circumferential engagement with a finger of a medical practitioner.

2. The device as set forth in claim 1, whereby the portions are integrally formed with the base at a proximal end of the base.

3. The device as set forth in claim 2, whereby the prongs are generally conically shaped.

4. The device as set forth in claim 1, further comprising a ridge positioned medially on the inner surface of the base.

5. The device as set forth in claim 4, further comprising a first knurl formed on an inner surface of the first portion and a second knurl formed on an inner surface of the second portion.
